(19) United States
(12) Patent Application Publication
(10) Pub. No.: US 2003/0077256 A1
(43) Pub. Date: Apr. 24, 2003

(54) Pancreas regeneration using embryonic pancreatic cells

(76) Inventors: Paul Czernichow, Paris (FR); Raphael Scharfmann, Paris (FR)

Correspondence Address: Norman H. Stepno, Esquire, BURNS, DOANE, SWECKER & MATHIS, L.L.P., P.O. Box 1404, Alexandria, VA, 22313-1404, US

(21) Appl. No.: 09/981,750

(22) Filed: Oct. 19, 2001

(51) Int. Cl. A01K 67/027; C12N 5/08; A61K 45/00

(52) U.S. Cl. 424/93.7; 435/366

(57) ABSTRACT

The invention provides a method of regenerating pancreas function in an individual by transplantation of an effective amount of functional pancreatic cells derived from embryonic pancreatic cells not older than 10 weeks of development. Also provided is the method of producing functional animal pancreatic cell.

The invention also provides a method of treatment of diabetics. Also are provided pancreatic beta cells as a medicament to treat diabetics.

### FIELD OF THE INVENTION

The invention relates to the filed of biology and in particular to the field of cellular biology and cellular therapy.

### BACKGROUND OF THE INVENTION

Type I diabetes is due to the destruction by immune mechanisms of pancreatic beta cells, resulting in the lack of insulin production and hyperglycemia. Cell therapy using beta cells from donors could represent one way to cure diabetic patients. However, two main problems have to be solved before this goal can be reached. First, immunosuppressive protocols have to be designed to provide immunologic protection of the graft. Recent reports indicate that progress has beer made in this field (Shapiro et al., 2000). The second point to be solved concerns the small number of mature beta cells from donors that are available for grafting (Weir and Bonner-Weir, 1997). There is a need to provide alternative sources of functional mature beta cells. That is the problem the present invention wishes to solve. During the last few years, it has been proposed that by understanding and recapitulating beta cell development that occurs during embryonic and fetal life, new beta cells could be produced that could be used for cell therapy of type I diabetes. Huge effort and progress have thus been made to define the molecular mechanisms that control prenatal pancreatic development in rodents and the role of specific transcription and growth factors has boon defined (Edlund, 1998; St Onge et al., 1999; Wells and Melton, 1999; Scharfmann, 2000; Grapin-Botton and Melton, 2000; Kim and Hebrok, 2001). Diffferent tissue sources potentially rich in precursor cells are also currently tested for their ability to differentiate into mature beta cells. Such cells derive either from fetal or neonatal porcine pancreas (Yoom et al., 1999; Otonkoski et al., 1999), or from fractions of human adult pancreas enriched in duct cells and that are thought to contain precursor cells (Bonner-Weir, 1997). So far, prenatal human pancreatic tissues (14-24 weeks) have been used unsuccessfully because all the tissues derived from fetuses were at late stages of development and were already quite mature when used in different assays (Tuch et al., 1984; Tuch et al., 1986; Sandla et al., 1985; Hayek et al., 1997; Goldrath et al., 1995). For instance, Tuch et al (1984), Sandler et al. (1985), Goldrath et al. (1995) and Fovlsen et al. (1974) used human pancreatic fragments of 14 to 24 weeks of development that have been engrafted into immunoincompetent mice with the goal of following endocrine tissue development. After a few weeks or months in recipient mice, all endocrine cell types were found when human tissues were removed (Tuch et al., 1984; Sandier et al., 1985). However, it is important to remember that between 14 and 24 weeks of development (the age of the tissue at the time of the transplantation), endocrine cells are already present and associated into islets of Langerhans (Bouwens et al., 1997; Stefan et al., 1983; Fi2kayama et al., 1986; Miettinen et al., 1992). Moreover, in these experiments using late human fetal tissues, when the quantity of insulin-expressing cells present in the graft was compared before and after transplantation, no clear increase in the beta cell mass was detected (Tuch et al.). It is consequently difficult to determine whether the human endocrine cells that were present after a few weeks or months in the mouse were newly formed endocrine cells or cells that existed before transplantation and did survive.

The present invention solves the above-mentioned problem of providing mature beta cells; indeed the inventors demonstrate that functional human beta cells can develop in NOD/scid mice from immature human embryonic pancreases not older than 10 weeks of development. More precisely, the inventors demonstrate that when human embryonic pancreases, that contained no or very few insulin-expressing cells (see FIG. 4), were engrafted into immunoincompetent mice, pancreatic tissue grew, its weight increasing 200 times within six months. At the same time, endocrine cell differentiation occurred, the absolute number of human beta cells being increased by a factor of 5,000. Finally, the endocrine tissue that developed was functional, being able to regulate the glycemia of mice deficient in rodent beta cells.

This model of development of human embryonic pancreases in NOD/scid mice that seem to mimic the ontogeny of the human pancreas that occurs in vivo, can now be used to study the mechanisms that control the development of the human embryonic pancreas, a question that has been partly eluded in the past due to the lack of proper experimental systems. Moreover, inventors' data do indicate that human embryonic pancreases represent a source of immature cells that can proliferate and differentiate in mass into beta cells when transplanted into an adult animal. This tissue may thus be useful as an alternative source of beta cells for transplantation.

### DETAILED DESCRIPTION OF THE INVENTION

The present invention provides a method of regenerating pancreas function in an individual, the method comprising

(a) introducing an effective amount of animal embryonic pancreatic cells not older than 10 weeks of development, into the kidney capsule of non-obese diabetic/severe combined immunodeficiency (NOD/scid) animal, excepted human, wherein said NOD/scid is of a different species than said animal from which are obtained said embryonic pancreatic cells; and

(b) allowing the animal embryonic pancreatic cells to develop, to differentiate and to regenerate at least a pancreatic function selected among the regulation of glycemia and the secretion of digestive enzymes; and

(c) transplantation of an effective amount of the animal functional pancreatic cells obtained at step (b), into said individual.

The term “individual” is a vertebrate, preferably a mammal. Mammals include, but are not limited to, humans, rodents (i.e. mice, rats, hamsters, farm animals, sport animals and pets. In a preferred embodiment, the invididual is a mammal and more preferably, a human.

In a preferred embodiment, the animal embryonic pancreatic cells are human embryonic pancreatic cells. Alternatively, it could be selected among, for instance, porcines, bovines, goats, sheep, primates, rodents (i.e. a mouse, a rat, a hamster . . . ), pancreatic cells. The animal embryonic pancreatic cells of the invention are cells that are selected among cells not older than 10 weeks, not older than 9 weeks, not older than 8 week, riot older than 7 weeks, not older than 6 weeks, not older than 5 weeks, not older than 4 week, not older than 3 weeks, not older than 1 week of development. In a preferred embodiment, the animal embryonic pancreatic cells of the invention is of 6 to 9 weeks of development. According to a preferred embodiment, the animal embryonic pancreatic cell of the invention is a human embryonic pancreatic cell that is not older than 9 weeks of development, more preferably comprised between 6 to 9 weeks of development.

The non obese diabetic/severe combined immunodeficiency (NOD/scid) animal is selected among bovines, porcines, horses, sheep, goats, primates excepted humans, rodents such as mice, rats, hamsters. In a preferred embodiment, the NOD/scid animal is a mouse. Preferably the NOD/acid mice of the invention is of any age of development, preferably sufficiently old to perform a graft into the kidney capsule. Preferably, the NOD/scid mice is about of the 2 to 15 weeks of development, more preferably to 6 to 8 weeks of development. A NOD/scid animal is an animal lacking T- and B-lymphocytes and failing to generate either humoral or cell-modiated immunity.

An “effective amount” is an amount sufficient to effect beneficial or desired clinical results. An effective amount can be administered in one or more applications, although it is preferable that one administration will suffice. For purposes of this invention, an effective amount of embryonic pancreatic cells is an amount that is sufficient to produce differentiated pancreatic cells which arc able to restore one or more of the functions of the pancreas. It is contemplated that a restoration can occur quickly by the introduction of relatively large numbers of pancreas cells, for example greater than 10⁹ cells. In addition, it is also contemplated that when fewer pancreatic cells are introduced, function will be restored when the pancreas cell or cells are allowed to proliferate in vivo. Thus, an “effective amount” of pancreatic cells can be obtained by allowing as few as one pancreas cell sufficient time to regenerate all or part of a pancreas. Preferably, an effective amount administered to the individual is greater than about 10¹ pancreas cells, preferably between about 10² and about 10¹⁵ pancreas cells and even more preferably, between about 10³ and about 10¹² pancreas cells. The effective amount of the animal pancreatic cells transplanted at step (c) of the method of the invention is more preferably between 10³ to 10¹² animal pancreatic cells. In terms of treatment, an “effective amount” of pancreatic cells is the amount which is able to ameliorate, palliate, stabilize, reverse, slow or delay the progression of pancreas disease, such as diabetics.

According Lo a preferred embodiment, the animal embryonic pancreas cells used in the methods of the present invention may be obtained from a heterologous donor (allograft), for example, an organ donor or a living donor. Alternatively, an autograft can be performed by removing a portion of an individual's pancreas at an early stage of development (prior to 10 weeks of development) or by reversing the differentiated phenotype of adult pancreas cells, and introducing the pancreas cells capable of regenerating pancreas function into the same individual. For autografts, at least about 5% of the donor individual's pancreas is removed. For allografts, at least about 5%, preferably greater than 30%, more preferably greater than 50% and even more preferably greater than 80% of the pancreas is removed. The method's of the present invention involve either allograft or autografts of pancreas cells. Each type of graft has its advantages. In particular, autografts (where pancreas cells from the same individual are used to regenerate pancreas function) is used to avoid immunological reactions. Graft versus host reactions occur when the donor and recipient are different individuals, and the donor's immune system mounts a response against the graft. Tissue typing and major histocompatibility (MHC) matching reduces the severity and incidence of graft versus host. Nonetheless, autologous introduction of pancreas cells will be especially useful in cases where the individual's pancreas is diseased. In such cases, a small amount of autologous embryonic pancreas tissue will regenerate a functional pancreas. Allografts are useful in cases where the pancras is not available, for instance if the pancreas of the individual is diseased. Various MHC matched pancreas cells can be maintained in vitro or isolated from donors and tissue typing performed to match the donor with the recipient. Immunosuppressive drugs, such as cyclosporin, can also be administered to reduce the graft versus host reaction. Allograft using the cells obtained by the methods of the present invention are also useful because a single healthy donor could supply enough cells to regenerate at least partial pancreas function in multiple recipients. Because the pancreas cells of the present invention are able to proliferate and differentiate so effectively, only a small number is required to repopulate a pancreas. Accordingly, one pancreas could be divided and used for multiple allografts. Similarly, a small number of cells from one pancreas could be culture in vitro and then used for multiple grafts. In an embodiment of the invention, the pancreatic cells of the invention cain be genetically modified in order they match all or various MHC (such cells constitute universal donor pancreatic cells). By pancreatic cells of the invention is meant either the embryonic pancreatic cells or the functional pancreatic cell that have developed and differentiate into the NOD/scid animal.

Suitable techniques for isolating pancreas tissue from a donor individual are known in the art. For example, extraction of pancreas cells through a biopsy needle or surgical removal of a portion or all of the pancreas tissue can be utilized.

Pancreatic tissue can be used in the methods of the present invention without further treatment or modification. Modifications are described below. For both modified and unmodified cells, it is preferred that single cell suspensions are obtained from the tissue. Cell suspensions can be obtained by methods known in the art, for example, by centrifugation and enzyme treatment. Pancreas tissue or cell suspensions can also be frozen and thawed before use. Preferably, the cells are fresh after isolation and processing.

Alternatively, the embryonic pancreatic cells of the present invention can be cultured long-term in vitro to produce stable lines of pancreas-regenerating cells. As used herein, the term “in vitro culture” refers to the survival of cells outside the body. Preferably, the cultures of the present invention are “long-term” cultures in that they proliferate stably in vitro for extended periods of time. These stable populations of cells are capable of surviving and proliferating in vitro with an embryonic pancreatic phenotype (i.e. these cells will be “stem” cells). Methods of culturing various types of stem cells are known in the art For example, WO 94/16059 describes long-term culture (greater than 7 months) of neuronal cells. Long-term culture of other types of stem cells are also described in the art and can be applicable to the cells of the present invention. The embryonic pancreatic cell cultured in vitro can be genetically modified to express a therapeutic gene.

According to the present invention, the animal functional pancreatic cells transplanted at step (c) are preferably introduced into the pancreas of said individual. Alternatively, such animal functional pancreatic cells are enclosed into implantable capsules that can be introduced into the body of an individual, at any location, more preferably in the vicinity of the pancreas, or the bladder, or the liver, or under the skin.

As used herein, the term <<introducing>> means providing or administering to an individual. In the present invention, functional pancreatic cells capable of regenerating functional pancreas cells are introduced into an individual. Methods of introducing cells into individuals are well known to those of skill in the art and include, but are not limited to, injection, intravenous or parenteral administration. Single, multiple, continuous or intermittent administration can be effected. The pancreas cells can be introduced into any of several different sites, including but not limited to the pancreas, the abdominal cavity, the kidney, the liver, the celiac artery, the portal vein or the spleen. Preferably, the pancreas cells are deposited in the pancreas of the individual.

Tho term “pancreas” refers to a large, elongated yellowish gland found in vertebrates. The pancreas has both endocrine and exocrine functions, producing the hormones insulin and glucagon and, in addition, secreting digestive enzymes such as trypsinogen, chymotrypsinogen. Procarboxypeptidase A and B, elastase, ribonuclease, desoxyribonuclease prophospholipase A, pancreatic lipase, pancreatic α-amylase The term “Pancreas cells” or “pancreatic cells” refers to cells obtained from the pancreas.

The present invention also provides a method wherein said individual is an insulin-dependent diabetic. Therefore, the invention also contemplated to provide a method of treatment of diabetes in a human patient in need of such treatment, the method comprising the steps of

(a) introducing an effective amount of human embryonic pancreatic cells not older than 10 weeks of development, more preferably from 6 to 9 weeks of development, into the kidney capsule of non-obese diabetic/severe combined immunodeficiency (NOD/scid) animal, excepted human; and

(b) allowing the embryonic pancreatic cells to develop, to differentiate and to regenerate at least the pancreatic function; and

(c) transplantation of an effective amount of the human functional pancreatic cells obtained at step (b), into said patient,

(d) and treating diabetics, wherein said treatment is effected by the regeneration of said pancreatic function of regulation of glycemia.

The previously described method is more specifically dedicated to the treatment of diabetes in a human patient.

As used herein, the term <<regeneration of said pancreatic function >> refers to the growth or proliferation of new tissue. In the present invention, regeneration refers to the growth and development of functional pancreas tissue. In most instances, the regenerated pancreas tissue will also have the cytological and histological characteristics of normal pancreas tissue For example, the pancreas cells introduced in to the individual and allowed to generate functional pancreas tissue are expected to express insulin and glucagon, and digestive enzymes along with other markers indicative of pancreas, such as Nkx6.1, Pax6, or PC⅓. Functions of the pancreas can be challenged by measures and tests known in the art, such as insulin or glucagon expression. According to a preferred embodiment, the non-obese diabetic/severe combined immunodeficiency animal is a mouse.

The present invention also provides a method of producing functional animal pancreatic cell wherein said method comprises the steps of

(a) introducing an effective amount of animal embryonic pancreatic cells not older than 10 weeks of development, more preferably from 6 to 9 weeks of development, into the kidney capsule of non-obese diabetic/severe combined immunodeficiency (NOD/scid) animal, excepted human, wherein said NOD/scid is of a different species than said animal from which are obtained said embryonic pancreatic cells; and

(b) allowing the animal embryonic pancreatic cells to develop, to differentiate and to regenerate at least a pancreatic function selected among tho regulation of glycemia, and the secretion of digestive enzymes; and

(c) collecting animal pancreatic cells obtained at step (b), and

(d) optionally in vitro culturing the cells obtained at step (c).

(e)optionally immortalizing the cells obtained at step (d).

The isolated cells can be cultured in vitro prior to introduction into the individual. Suitable culture media are well known to those of skill in the art and may include growth factors or other compounds which enhance survival, proliferation or selectively promote the growth of certain sub-types of pancreatic cells such as alpha, beta, delta pancreatic cells.

The present invention also provides a method wherein said functional animal pancreatic cell is further genetically modified.

Before introduction into an individual, the isolated functional pancreas cells of the present invention can he further modified, for example, using particular cell culturing conditions or by genetic engineering techniques. This modification includes the introduction of a therapeutic gene into said cell, either integrated into the genome of said cell, or present as an extrachromosomal replicon. A “therapeutic gene” is a gene that corrects or compensates for an underlying protein deficit or, alternately, that is capable of down-regulating a particular gene, or counteracting the negative effects of its encoded product, in a given disease state or syndrome. Moreover, a therapeutic gene can be a gene that mediated cell killing, for instance, in the gene therapy of cancer. According to another embodiment, the transposable DNA sequence of interest is a reporter gene as previously defined.

Genetic engineering techniques can be used to introduce therapeutic genes to be expressed. The invention also encompasses treatment of diseases or amelioration of symptoms associated with disease, amenable to gene transfer into pancreas cell populations obtained by the method disclosed herein. Diseases related to the lack of a particular secreted product including, but not limited to, hormones, enzymes, interferons, growth factors, or the like can also be treated by genetically modified pancreas cells.

The therapeutic gene is transduced into the cell by any number of methods, e.g., using naked polynucleotides (e.g., by electroporation) or using delivery systems such as adenoviral vectors, adeno-associated viral vectors, retroviral and liposomes. Direct physical methods also are available. These methods include the use of the “gene gun” or calcium phosphate transfection method. As noted above, any method of gene transfer is encompassed by this invention.

Moreover, the present invention provides a functional animal pancreatic cell obtained by the method of the invention wherein said cell are selected among pancreatic alpha cell, pancreatic beta cells, pancreatic delta cells. Preferably, said cell is a pancreatic beta cell, and more preferably, it is a human pancreatic beta cell. Said pancreatic beta cell is functional and expresses insulin in response to glucose. Moreover, the present invention provides a functional pancreatic beta cell that expresses glucagon in response Lo glucose. Additionally, said functional pancreatic cell expresses and secretes digestive enzymes. Said cell is preferably a human cell.

The present invention also provides a functional animal pancreatic cell obtained by the method of the invention wherein said cell is immortalized with a virus or a variant or a fragment thereof, said virus being selected among retrovirus, more precisely, lentivirus, Simian virus 40 (SV40) and Epstein-Bahr virus.

It is another embodiment of the present invention to provide a pancreatic cell of the invention as a medicament to perform cell therapy. More precisely, the present invention relates to the use of a pancreatic cell of the invention for preparing a medicament to treat diabetics, hypoglycemia, or pathologies associated to a dysfunction of the digestive enzymes. In a preferred embodiment, the invention relates to the use of a pancreatic cell for preparing a medicament to treat diabetics.

The present invention also provides the use or a pancreatic cell of the invention for cell therapy.

It is also a goal of the present invention to use a pancreatic sell of the invention for studying the physiopathological development of diabetes. Such a cell in vitro cultured or engraft into an individual as an allograft or an autograft, would be highly useful to study molecular, biological, biochemical, physiological and/or physio-pathological mechanisms of glycemia regulation and/or also digestive enzyme expression, secretion and regulation.

The present invention also provides a method of producing animal pancreatic cell at different stages of development wherein said method comprises the steps of:

(a) introducing an effective amount of animal embryonic pancreatic cells not older than 10 weeks of development, into the kidney capsule of non-obese diabetic/severe combined immuno-deficiency (NOD/scid) animal, excepted human, wherein said NOD/scid is of a different species than said animal from which are obtained said embryonic pancreatic cells; and

(b) allowing the animal embryonic pancreatic cells to develop, optionally to differentiate and optionally to regenerate at least a pancreatic function selected among the regulation of glycemia and secretion of digestive enzymes, and

(c) collecting animal pancreatic cells obtained at step (b) at different periods of time, and

(d) optionally in vitro culturing the cells obtained at step (c).

(e) optionally immortalizing the cells obtained at step (d).

Such pancreatic cells obtained by the method of the invention are useful for studying pancreas development.

Another embodiment of the present invention is the NOD/scid animal in which the embryonic pancreatic cells have been engrafted.

Such NOD/scid animal comprises at least one functional pancreatic cell of the invention at any stage of development, which is derived from the engrafted embryonic pancreatic cell.

The present invention relates to the use of the NOD/scid animal of the invention to study and understand the development and the functioning of healthy or pathologic pancreas. Such animal constitutes an excellent model to understand and study pancreatic development, mainly human pancreatic development.

Moreover, such animal would be useful to screen compounds able to modulate pancreas development or to modulate the regulation of glycemia, by modulating or by acting, for instance, on the insulin or glucagon expression, or on the expression of any targeted gene or protein involved in glycemia regulation. Such animal would be also useful to screen compounds able to modulate the expression of digestive enzymes. By “modulate”, it is meant “enhance”, “decrease”, or “cancel”.

Unless defined otherwise, all technical and scientific terms used herein have the same meaning as is commonly understood by one of the skill in the art to which this invention belongs.

The figures and examples presented below are provided as further guide to the practitioner of ordinary skill in the art and are not to be construed as limiting the invention in anyway.

### BRIEF DESCRIPTION OF THE FIGURES

FIG. 1 Development of the human pancreas in NOD/scid mice. (A) a pancreas at 8 weeks of development before transplantation. (B-E) the pancreases were grafted under the kidney capsule of NOD/scid mice and analyzed 7 days (B), 2 months (C), 6 months (D)), and 9 months (E) later.

FIG. 2: Evolution of the weight of the transplanted pancreas.

Grafts were removed at different time points after transplantation, finely dissected to remove the tat, and weighed. A total of 22 graft were analyzed.

FIG. 3: Histological analysis. Human pancreas at 8 weeks of development before transplantation (A), and one month (B) and six months (C) after transplantation stained with an anti-pan cytokeratin antibody (revealed in green) or with an anti-vimentin antibody (revealed in red).

FIG. 4: Development of the pancreatic endocrine tissue in NOD/scid mice. Eight-week pancreas before grafting (A), and 7 days (B), one month (C), 2 months (D), 6 months (E) and 9 months (F) after transplantation.

Insulin (revealed in green) and glucagon (revealed in red) immunostainings. The arrows in (A) represent 2 cells that stain positive for both insulin and amylase. In G and H are shown representative in hybridizations of a proinsulin probe on sections from 8-week human pancreas before grafting (G) and after 6-months engraftment (H).

FIG. 5: Evolution of the endocrine cell mass during the transplantation period.

The absolute mass of insulin-expressing cells is presented in arbitrary units. A total of 16 grafts were analyzed.

FIG. 6: Cell proliferation analysis.

Double immunostaining for BrdU (red) and pan-cytokeratin (green) (A, C) or BrdU (red) and insulin (green) (B, D) on sections of human embryonic pancreas that developed in scid mice during 1 month (A and 1) or during 3 months (C, D) Mice were sacrificed 2 hours after BrdU injection.

FIG. 7 human endocrine cells developed in mice resemble mature endocrine cells.

Sections of a human embryonic pancreas 6 months after transplantation. (A). insulin (revealed in green) and Pax 6 (revealed in red); (b). Insulin (revealed in green) and Nkx6.1 (revealed in red); (C, D). Insulin (revealed in red) and PC⅓ (revealed in green); (E). Insulin (revealed in green) and Cytokeratin-19 (revealed in red); (F). Cytokeratin 19 alone (revealed in red).

FIG. 8: Functional development of the human pancreas graft.

(A) Three months after transplantation, scid mice (red lines) were injected with alloxan. Non-qrafted mice (blue line) also received alloxan. While the glycemnia of the non-grafted mice increased rapidly, that of the grafted mice remained stable. When grafts were removed by nephrectomy at day 7 or day 43, glycemia increased rapidly.

(B) Mouse pancreas before alloxan treatment and at the end of the experiments (C) (day 43 after alloxan) stained for insulin (revealed in red) and glucagon (revealed in green), indicating that alloxan has destroyed the vast majority of host-insulin-expressing cells.(D). Section of the human graft at the end of the experiment (day 43 after alloxan) stained for insulin (revealed in red) and glucagon (revealed in green), indicating that alloxan had no effect on human beta cells that developed.

### EXAMPLES

1—RESEARCH DESIGN AND METHODS

1.1) Human tissues

Human pancreases were dissected from embryonic tissue fragments obtained immediately after voluntary abortions performed between 6 and 9 weeks of development (WD), in compliance with the current French legislation and the guidelines of our institution. The warm ischemia time was less than 30 min. Gestational ages were determined from several developmental criteria: duration of amenorrhea; crown-rump length measured by ultrasound scan; hand and foot morphology. Pancreases were dissected and either fixed and embedded in paraffin or grafted to non-obese diabetic/severe combined immunodeficiency (NOD/scid) mice as described below.

1.2) Animals and transplantation into NOD/acid mice

NOD/scid mice were bred in isolators supplied with sterile-filtered, temperature-controlled air. Cages, bedding and drinking water were autoclaved. Food was sterilized by X-ray irradiation. All manipulations were performed under a laminar flow hood. Embryonic pancreases (6-9 weeks of development (WD)) were implanted, using a dissecting microscope, under the let kidney capsule of 6- to 8-week-old NOD/scid mice that had been anesthetized with Hypnomidate (Janssen-Cilag). At different time points after the graft (7 days - 9 months), mice were sacrificed and the grafts were removed, weighed, fixed in formalin 3.7% and embedded in paraffin. For cell proliferation analysis, mice were injected with Bromo-deoxy Uridine (BrdU) (50 mg/kg) 2 hours before sacrifice.

1.3) Immunohistochemistry.

Four μm-thick sections were cut on gelatinized glass slides. For immunostaining, sections were deparaffinized in toluene, rehydrated, microwaved in citrate buffer 0.01 M, pH 6, and permeabilized for 20 min in Tris-Buffered Saline (TBS) containing 0.1% Triton. Non-specific sites were blocked for 30 min in TBS containing 3% BSA and 0.1% Tween 20 and sections were incubated overnight at 4° C. with primary antibodies. The sections were then washed and incubated 1 h at room temperature with the appropriate secondary antibodies, labeled with 2 different fluorochromes. The primary antibodies were; mouse anti-human insulin (Sigma Aldrich, 1/1000) guinea pig anti-pig insulin (Dako; 1/2000); mouse anti-human glucagon (Sigma Aldrich, 1/2000); rabbit anti-human pan-cytokeratin (Dako, 1/500); mouse anti-human cytokeratin 19 (Dako; 1/50); mouse anti-pig vimentin (Dako; 1/30); rabbit anti-proconvertase ⅓ (gift from Dr Steiner, 1/200); rabbit anti-Pax6 (gift from Dr S. Saule); rabbit anti-rat Nkx6.1 (gift from Dr Serup); Mouse anti-BrdU (Amersham). Fluorescent secondary antibodies werc: fluorescein-anti-guinea pig antibodies (Dako, 1:500); fluorescein-anti-rabbit antibodies (Immunotech, 1/200); fluorescein-anti-mouse antibodies (Immunotech, 1/200); Texas-red-antimouse antibodies (Jackson, 1/200); Texas-red-anti-rabbit antibodies (Jackson, 1/200).

1.4) Surface quantification and statistical analysis.

All images were numerized using a Hamamatsu C5810 cooled tri-CCD camera. Pictures were made at tho same magnification, and analyzed with the IPLab software (version 3.2.4, Scananalytics Inc.). For each transplanted tissue, sections were taken at regular intervals throughout the graft and stained for insulin. Three to four sections and 3-5 views per section were analyzed. The evolution of the beta cell mass during the transplantation period was calculated as the product of the surface that stained positive for insulin by the corresponding graft weight.

1.5) In situ hybridization.

For in situ hybridization, sections were deparrafinized, rehydrated and permeabilized in PBS containing 1% Triton X-100. Prehybridization was dope at 70° C. in hybridization buffer (50% formamide, 5 X SSC, 5X Denhardts' solution, 250˜μg/ml yeast RNA, 500 μg/ml herring sperm DNA). RNA probes were labeled with DIG-UTP by in vitro transcription using the DIG-RNA labeling kit (Boehringer Mannheim). hybridization was initiated by addition of fresh hybridization buffer containing Ipg/ml probe and continued overnight at 70° C. Thereafter, the slides were washed with decreasing concentrations of SSC. Revelation was processed by immunohistochemistry. Non-specific sites were blocked with 2% blocking reagent (Boehringer Mannheim) in Tris 25 mM pH7.5, NaCl 140 mM, KC1 2.7 mM Tween 20 0.1% for 30 min at room temperature. Slides were then incubated overnight at 4° C. with alkaline phosphatase-conjugated polyclonal sheep anti-DIG antibody (diluted 1:1000, Boehringer Mannheim). Tho reaction product was visualized by an enzyme-catalyzed color reaction using nitro blue tetrazolium and 5-bromo-4chloro-3-indolyl-phosphate medium (Bohringer Mannheim). Sections were incubated until the colored reaction product developed at the sites of hybridization. The slides were washed in H₂O, mounted and visualized on a Leitz DMRD light microscope (Leica). The probe used here corresponded to human proinsulin.

1.6) Test of induction of diabetes.

To determine the capacity of the graft to regulate the glycemia of the mouse, grafted and nongrafted NOD/scid mice were injected intravenously (i.v.) with alloxan (Sigma-Aldrich, 90 mg/kg body weight), that is known to destroy rodent, but not human, beta cells (Eizirik et al., 1994). Glucose levels were measured on blood collected from the tail vein every day during one week, using a portable glucose meter (GlucoMen, A. Menarini diagnostics, Firenze, Italy). To confirm the contribution of the graft to the normalization of blood glucose values in the host, grafts were removed by unilateral nephrectomy at different time points (7 days or 42 days) after the injection of alloxan and blood glucose levels were measured.

2 —RESULTS

2.1) Human embryonic engraftment in NOD/scid mice.

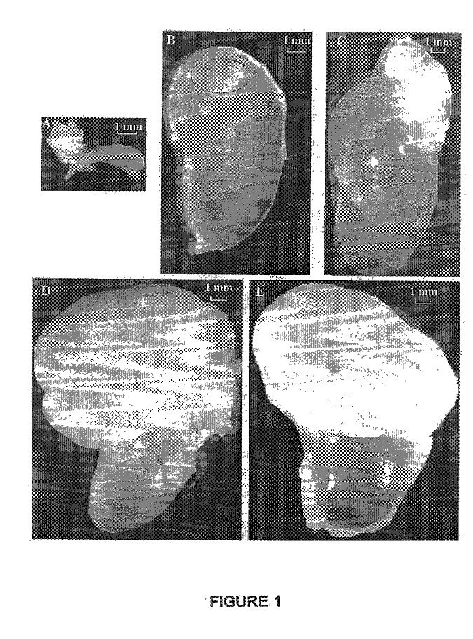

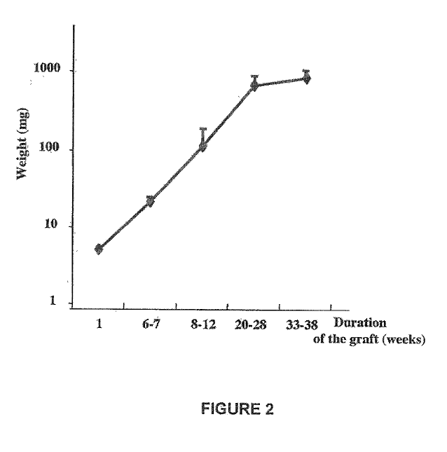

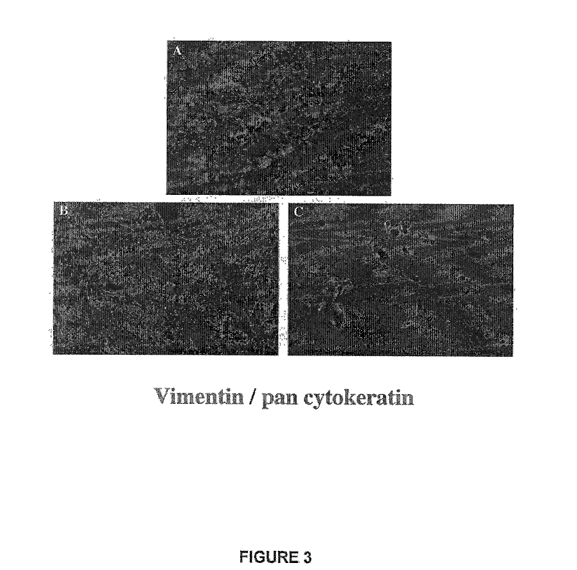

In the present study, 48 embryonic pancreatic tissues (6-9WD) were grafted to NOD/scid mice. Mice were sacrificed at different time points after transplantation (7 days-9 months), and the grafts were dissected. Thirty-nine grafts were recovered. Among the 9 non-recovered grafts, one grafted mouse died, but the graft was present. In 8 cases, mice died for unknown reasons and graft growth was not analyzed. The evolution of grafted tissues in terms of volume and mass was next followed. As shown in FIG. 1, the human embryonic tissue developed massively when grafted under the kidney capsule of the scid mice. While at 7-9 weeks of gestation, before grafting, the volume of embryonic pancreas was not more than 4 mm³ (FIG. IA), it increased with time in the mouse host and reached a volume of a few cm³ 6 months later (FIG. 1B-E). The evolution of the grafted tissue in term of mass was also followed. While, after 1 week in the mouse, graft weight was less than 10 mg, in the same range of the ungrafted tissue, it next increased rapidly with time to reach 100 mg after 8-12 weeks and 1000 mg after 33-38 weeks (FIG. 2). Immunohistochemistry using anti-cytokeratin and anti-vimentin antibodies was performed to follow the evolution during the grafting period of the epithelial and mesenchymal cells present in the human embryonic pancreas before transplantation. As shown in FIG. 3A, before grafting, an 8WD pancreas is composed of epithelial cells forming ducts and mesenchymal cells. One month and 6 months after transplantation, the tissue was also composed of epithelial cells that stained positive for cytokeratin and mesenchymal cells positive for vimentin, indicating that both cell types did develop during the graft (FIG. 3B and C).

2.2) Development of tho endocrine tissue.

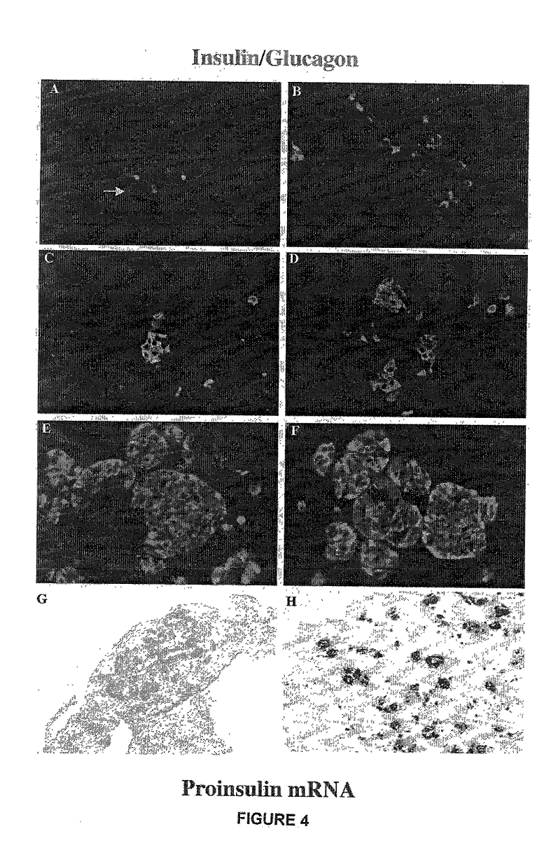

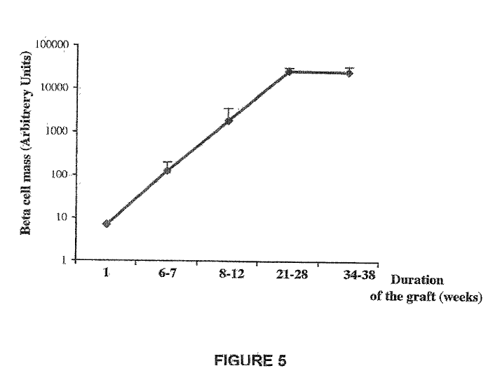

Before transplantation, only a few endocrine cells were detected by immunohistochemistry that stained positive either for glucagon, or for both insulin and glucagon. Such cells were dispersed in the pancreatic tissue and were not associated into islets of Langerhans (FIG. 4A). Once transplanted, the endocrine tissue started to develop and the number of endocrine cells increased with time (FIG. 48-F). In panels G and H are shown representative hybridizations for proinsulin before and after transplantation for 6 months of an 8-week old pancreas. Huge increase in the number of cells that express proinsulin mRNA can be clearly visualized. Evolution of the insulin-positive cell mass was quantified after immunohistochemistry. As shown in FIG. 5, the absolute surface occupied by insulin-expressing cells was multiplied by 300 after 8-12 weeks in the mouse and by 5,000 after 21-28 weeks.

2.3) Human undifferentiated epithelial cells, but not endocrine cells, proliferated during the engraftment period.

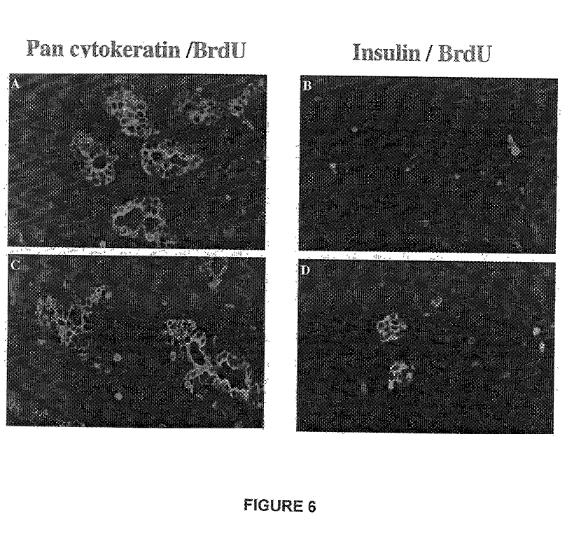

To define whether the increase in the absolute number of endocrine cells was due to the differentiation of precursor cells or to the proliferation of the few endocrine cells that were present before grafting, engrafted mice were injected with BrdU 2 hours before sacrifice and immunohistological analysis was performed. As shown in FIG. 6, after both 1 and 3 months of development in the mouse, while cells that stained positive for both cytokeratin and BrdU were frequently detected, cells positive for both insulin and BrdU were very rarely found. These results strongly suggest that increase in the endocrine cell mass was duo to the differentiation of precursor cells, rather than to the proliferation of rare preexisting endocrine cells.

2.4) Human endocrine cells developed in mice resemble mature endocrine cells.

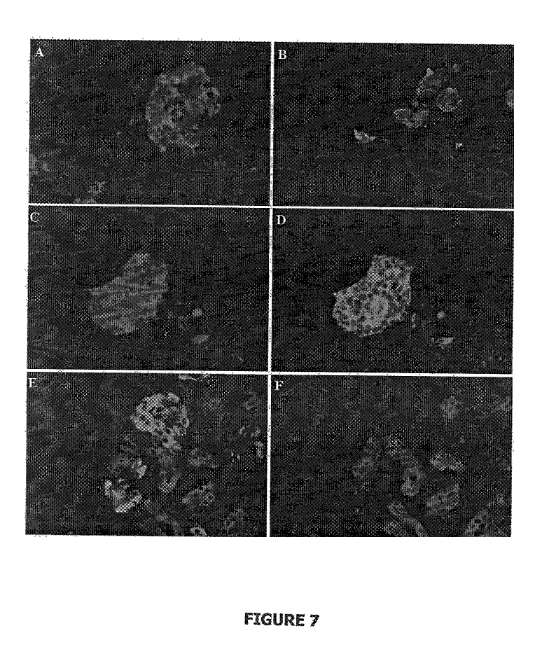

To define whether human endocrine cells that developed in NOD/scid mice express markers known to be present in human beta cells developed in vivo, a series of antibodies were tested by immunohistochemistry As shown in FIG. 7 beta cells developed in NOD/scid mice express specific transcription factors such as Nkx6.1 and Pax6 (Panels A, B), as well as PC ⅓, an enzyme necessary for the processing of proinsulin into insulin (panels C, D). Moreover, endocrine cells in the grafts are frequently associated in islets of Langerhans, with a core of insulin-expressing cells surrounded by glucagon-expressing cells (FIG. 4, panels E, F) Finally, while as described previously (Bouwens et al. 1997), the first endocrine cells found in human embryonic pancreas stain positive for cytokeratin, the human insulin-expressing cells that developed in NOD/scid mice did not express cytokeratin 19 (FIG. 7, panels E, F).

2.5) Functional development of human pancreas grafts.

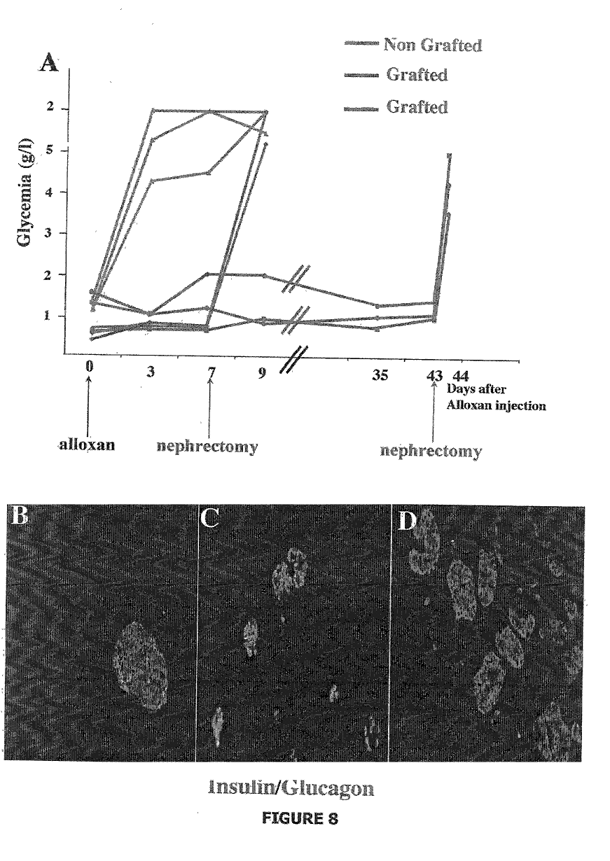

To define whether the human beta cells that developed in the grafts were functional, 15 NOD/Acid mice wore injected with human embryonic pancreas. Three months later, grafted or non-grafted NOD/scid mice were injected with alloxan, a drug known to be toxic for murine, but not for human beta cells (Eizirik et al., 1994). Before alloxan injection, blood glucose levels were not statistically different in the transplanted and non-transplanted mice. After alloxan treatment, the glycemia of all non-grafted mice increased up to 6 g/l. Conversely, the glycemia remained stable in 12 omit of 15 engrafted mice. To demonstrate that glycemia regulation in engrafted mice injected with alloxan is indeed due to the development of the graft, unilateral nephrectomies were performed to remove the grafts in 6 mice and blood glucose levels were monitored. As shown in FIG. 8A, after removal of the graft by unilateral nephrectomy, either 7 or 43 days after alloxan injection the mice became hyperglycemic. Murine pancreases and grafts were also analyzed for the presence of insulin- and glucagon-expressing cells before alloxan injection, or at the end of the experiments when the animals were sacrificed. As shown in FIG. 8, very few insulin-expressing cells were detected in the murine pancreas of grafted NOD/scid mice that had been injected with alloxan, compared to NOD/scid mice that were not treated with alloxan. On the other hand, a huge amount of insulin-producing cells was present in the human graft after alloxan treatment.

The inventors demonstrate that human early embryonic pancreas can develop when engrafted under the kidney capsule of NOD/scid mice. The size and weight of the grafts increased considerably and endocrine cells differentiated which were organized into islets of Langerhans and showed numerous criteria of maturity. Finally, the human endocrine pancreatic tissues that developed could reverse diabetes in mice, indicating its functionality.

In the present study, tho inventors used NOD/scid mice as recipients for transplantation. NOD/scid mice were generated by crossing the said mutation from C.B-17-scid/scid mice onto the NOD background. These animals are lacking T- and B-lymphocytes, (Shultz et al., 1995), and fail to generate either humoral or cell-mediated immunity. Because, of the absence of xenograft rejection in scid mice, they were previously used as recipients for human or fetal hematolymphoid tissues and cells (Roncarolo et al., 1995). Non hematopoietic human tissues such as ovarian cortex (Weissman et al., 1999), thyroid (Martin et al., 1993), skin (Levy et al., 1998) and airway (Delplanque et al., 2000) were also successfully transplanted in this model. However, the capacity of these tissues to develop functional properties and to replace a physiological function has been demonstrated only rarely. One example of physiological function replacement is the demonstration that adrenocortical tissue can form by transplantation of bovine adrenocortical cells and replace the essential functions of the mouse adrenal gland (Thomas et al ., 1997). However, in this work, the transplanted tissue was not of human but of bovine origin. Moreover, the tissue had been expanded from a primary culture of bovine adrenocortical cells. Donor cells were thus already fully differentiated at the time of grafting.

In the present invention, the inventors demonstrate that immature human embryonic pancreas can develop and acquire functional properties in scid mice.

By grafted immature rudiments that contained undifferentiated epithelial cells and mesenchymal tissue and almost no insulin-expressing cells, the inventors demonstrate that the absolute mass of insulin-expressing cells was multiplied by nearly 5,000 after 6 months in the mouse. The fact that very few endocrine cells were present before transplantation, while a massive amount of endocrine cells was detected a few weeks later, clearly indicates that neoformation of endocrine cells occurred in the present model.

Theoretically, the observed increase in the human beta sell mass could be due either to the proliferation of rare preexisting insulin-expressing cells, or to the differentiation of precursor cells. It is thought that during prenatal life, increase in the beta cell mass in mainly due to the differentiation of precursor cells rather than to the proliferation of preexisting beta cells. This is quite clear in rodents where A large number of experiments have been performed that indicate that the increase in the endocrine cell mass observed during fetal life cannot be explained by the proliferation of preexisting endocrine cells (Swenne, 1992)). While less information is available, this seems to be also the case in humans, where, during embryonic/fetal life, insulin-expressing cells stain rarely positive for Ki67, and hence are rarely or not cycling (Potak et al., 2000; Bouwens et al., 1997). The inventors' data indicate that when chimeric mice are injected with BrdU, a large number of cytokeratin-positive cells present in the graft stain positive for BrdU, while no or very rare insulin-positive cells do. Thus, it can be postulated that in the grafts newly formed beta cells derived from precursor cells present in the duct epithelium that did proliferate and differentiate during engraftment rather than from proliferation of the few endocrine cells present in the rudiment, a mechanism that does recapitulate normal development.

Different arguments indicate that the human beta cells that did develop in vivo in NOD/scid mice are mature. First, these insulin-expressing cells did not coexpress glucagon and arc thus different from the first insulin-expressing cells detected in the human pancreas at early stages of development (Polak et al., 2000; Larsson and Ilougaard, 1994; De Krijger et al., 1992). Next, it has been shown that the, insulin expressing cells present in the pancreas before 16 weeks of development express cytokeratin 19, while the insulin-expressing cells found later during development stain negative for this marker (Bouwens et al., 1997). The inventors' data indicate that the human beta cells that develop in NOD/scid mice stain negative for cytokeratin 19 and do thus resemble adult mature beta cells. Next, human beta cells that develop in NOD/scid mice express the prohormone convertase PC 1/PC3, an enzyme that is necessary for the processing of proinsulin into insulin (Kaufman et al., 1997; Furuta et al., 1998). Finally, the inventors' data indicate that the human endocrine cell mass that developed in NOD/scid mice is able to perfectly regulate the glycemia of NOD/scid mice deficient in endogenous beta cells, and hence is functional. These human endocrine cells remain functional and can regulate the glycemia of the mice for at least 43 days, the longest period tested before removing the graft.

The inventors demonstrate here that newly differentiated human beta cells that are able to regulate the glycemia of the host deprivated of its own beta cells can be produced from human early embryonic pancreas. Human embryonic pancreas does thus represent an alternative source of tissue useful to generate functional human beta cells for transplantation, Moreover, the model of mice grafted with human embryonic pancreas can now be used to progress in the study of the development of the human pancreas, a type of study that were difficult to perform due to the lack of human embryonic pancreases and of proper experimental systems.

### REFERENCES

Bonner-Weir et al. (2000) Proc Natl Acad Sci (USA 97:7999-8004

Bouwens et al. (1997) Diabetologia 40:398-404

De Krijger et al. (1992) Dev siol 153:368-375

Delplanque et al. (2000) J Cell Sci 113:767-778

Edlund (1998) Diabetes 47:1817-1823

Eizirik et al. (1994) Proc Natl Acad Sci USA 91:9253-9256

Fukayama et al. (1986) Differentiation 31:127-133

Goldrath et al. (1995) Transplantation 59:1497-1500

Grapin-Botton, Melton (2000) Trends Genet 16:124-130

Hayek, Beattie (1997) J Clin Endocrinol Metab 82:2471-2475

Kaufmann et al. (1997) Diabetes 46:978-982

Kim, Hebrok (2001) Genes Dev 15:111-127

Larsson, Hougaard (1994) Endocrine 2:759-765

Levy et al. (1998) Gene Ther 5:913-922

Martin et al. (1993) J Clin Endocrinol Metab 77:305-310

Miettinen, Heikinheimo (1992) Development 114:833-840

Otonkoski (1999) Transplantation 68:1674-1683

Polak et al. (2000) Diabetes 49:225-232

Povlsen et al. (1974) Nature 248:247-249

Roncarolo et al. (1995) Georgetown TX:Landes

Sandler et al. (1985) Diabetes 34:1113-1119

St-Onge et al. (1999) Curr Opin Genet Dev 9:295-300

Shapiro et al. (2000) N Engl J Med 343:230-238

Sharfmann (2000) Diabetologia 43:1083-1092

Shultz et al. (1995) J Immunol 154:180-191

Stefan et al. (1983) Diabetes 32:293-301.

Swenne (1992) Diabetologia 35:193-201

Thomas et al. (1997) Nat Med 3:978-983

Tuch et al. (1984) Diabetes 33:1180-1187

Tuch et al. (1986) Diabetes 35:464-469

Weir, Bonnor (1997) Diabetes 46:1247-1256

Weissman et al. (1999) Biol Reprod 60:1462-1467

Wells, Melton (1999) Annu Rev Cell Dev Biol 15:393-410

Yoon et al. (1999) Cell Transplant 8:673-689

1. A method of regenerating pancreas function in an individual, the method comprising
(a) introducing an effective amount of animal embryonic pancreatic cells not older than 10 weeks of development, into the kidney capsule of non-obese diabetic/severe combined immunodeficiency (NOD/scid) animal, excepted human, wherein said NOD/ scid is of a different species than said animal from which are obtained said embryonic pancreatic cells; and
(b) allowing the animal embryonic pancreatic cells to develop, to differentiate and to regenerate at least a pancreatic function; and
(c) transplantation of an effective amount of the animal functional pancreatic cells obtained at step (b), into said individual.

2. The method of claim 1, wherein the individual is a mammal.

3. The method of claim 2, wherein the mammal is a human.

4. The method of claim 1, wherein said animal embryonic pancreatic cells are human embryonic pancreatic cells.

5. The method of claim 4, wherein said human embryonic pancreatic cells is of 6 to 9 weeks of development.

6. The method of claim 1, wherein the non-obese diabetic/severe combined immunodeficiency animal is a mouse.

7. The method of claim 1, wherein the effective amount of the animal pancreatic cells transplanted at step (c) is comprised between about 10³ to about 10¹² animal pancreatic cells.

8. The method of claim 1, wherein the animal pancreatic cells transplanted at step (c) are introduced into the pancreas of said individual.

9. The method of claim 1, wherein said pancreatic function is the regulation of glycemia.

10. The method of claim 1, wherein said individual is an insulin-dependant diabetic.

11. A method of treatment of diabetes in a human patient in need of such treatment, the method comprising the steps of:
(a) introducing an effective amount of human embryonic pancreatic cells not older than 10 weeks of development, into the kidney capsule of non-obese diabetic/severe combined immunodeficiency (NOD/scid) animal, excepted human; and
(b) allowing the embryonic pancreatic cells to develop, to differentiate and to regenerate at least the pancreatic function; and
(c) transplantation of an effective amount of the human functional pancratic cells obtained at step (b), into said patient,
(d) and treating diabetics, wherein said treatment is effected by the regeneration of said pancreatic function of regulation of glycemia.

12. The method of claim 11, wherein the non-obese diabetic/severe combined immunodeficiency animal is a mouse.

13. A method of producing functional animal pancreatic cell wherein said method comprises the steps of:
(a) introducing an effective amount of animal embryonic pancreatic cells not older than 10 weeks of development, into the kidney capsule of non-obese diabetic/severe combined immunodeficiency (NOD/scid) animal, excepted human, wherein said NOD/scid is of a different species than said animal from which are obtained said embryonic pancreatic cells; and
(b) allowing the animal embryonic pancreatic cells to develop, to differentiate and to regenerate at least a pancreatic function; and
(c) collecting animal pancreatic cells obtained at step (b), and
(d) optionally in vitro culturing the cells obtained at step (c).
(e)optionally immortalizing the cells obtained at step (d)

14. The method of claim 13, wherein said pancreatic function is the regulation of glycemia.

15. Method according to claim 13 wherein said functional animal pancreatic cell is further genetically modified.

16. Functional animal pancreatic cell obtained by the method according to claim 13 wherein said cell is a pancreatic beta cell.

17. Pancreatic beta cell of claim 1, wherein said cell express insulin in response to glucose.

18. Functional animal pancreatic cell obtained by the method according to claim 13 wherein said cell is immortalized with a virus or a variant or a fragment thereof, said virus being selected among lentivirus, Simian virus 40, Epstein-Bahr virus.

19. Pancreatic cell according to claims 16 to 18 wherein said cell is a human cell.

20. Pancreatic cell according to claim 19 as a medicament.

21. Use of a pancreatic cell according to claim 19 for preparing a medicament to treat diabetics.

22. Use of a pancreatic cell according to claim 19 for cell therapy.

23. Use of a pancreatic cell according to claim 19 for studying the physiopathological development of diabetics.

24. A method of producing animal pancreatic cell at different stages of development wherein said method comprises the steps of:
(a) introducing an effective amount of animal embryonic pancreatic cells not older than 10 weeks of development, into the kidney capsule of non-obese diabetic/severe combined immunodeficiency (NOD/scid) animal, excepted human, wherein said NOD/scid is of a different species than said animal from which are obtained said embryonic pancreatic cells; and
(b) allowing the animal embryonic pancreatic cells to develop, optionally to differentiate and optionally to regenerate at least a pancreatic function; and
(c) collecting animal pancreatic cells obtained at step (b) at different periods of time, and
(d) optionally in vitro culturing the cells obtained at step (c).
(e) optionally immortalizing the cells obtained at step (d).

25. Use of pancreatic cells obtained by the method of claim 24 for studying pancreas development
